(19) United States
(12) Patent Application Publication
(10) Pub. No.: US 2025/0389689 A1
(43) Pub. Date: Dec. 25, 2025

(54) MICROFLUIDIC SENSOR FOR BACTERIA DETECTION IN BIOFLUIDS

(71) Applicant: UNIVERSITY OF MARYLAND, BALTIMORE COUNTY, Baltimore, MD (US)

(72) Inventors: Chengpeng CHEN, Columbia, MD (US); Tao ZHANG, Ellicott City, MD (US); Zumar MEHER, Ashton, MD (US); Curtis G. JONES, Baltimore, MD (US)

(21) Appl. No.: 19/243,936

(22) Filed: Jun. 20, 2025

Related U.S. Application Data
(60) Provisional application No. 63/663,163, filed on Jun. 23, 2024.

(51) Int. Cl. G01N 27/333; C12Q 1/02; G01N 27/327

(52) U.S. Cl. CPC G01N 27/333; C12Q 1/02; G01N 27/327

(57) ABSTRACT

A microfluidic device and method of using same, wherein the microfluidic device can trap bacteria from a biofluid, e.g., urine, and a silver ion selective electrode sensor can detect the loss of Ag⁺ in the microfluidic device because of the trapped bacteria. By knowing the loss of Ag⁺, the number of bacteria present in the biofluid can be accurately determined.

### CROSS REFERENCE TO RELATED APPLICATIONS

This application claims priority to U.S. Provisional Patent Application No. 63/663,163 filed on Jun. 23, 2024 in the name of Chengpeng CHEN et al. entitled “MICROFLUIDIC SENSOR FOR BACTERIA DETECTION IN BIOFLUIDS,” which is hereby incorporated by reference herein in its entirety.

### FIELD

The present invention relates to microfluidic device and method of using same to accurately determine the number of bacteria present in a biofluid.

### BACKGROUND

Bacterial infections pose a significant threat to human health, with urinary tract infection (UTI) being a common and problematic example. UTIs can lead to various health issues, including fever and dysuria, and are a frequent cause of acute illness and hospital admissions, particularly in children. Pediatric renal scarring associated with UTIs can result in long-term complications such as hypertension, pre-eclampsia, and renal failure. The majority of UTIs are most often caused by extraintestinal pathogenic Escherichia coli.

Traditionally, UTI diagnosis relied on the presence of over 105 colony-forming units (CFUs) of bacteria per milliliter of urine. However, as more patients presented symptoms with lower CFUs, the diagnostic threshold was reduced to 10³ CFU/mL. The conventional method for quantifying bacteria CFUs in urine involves plating the sample on agar media and manually counting the resulting colonies after 24 to 48 hours of culture. While effective, this process is time-consuming and requires specific facilities and trained personnel. Dipsticks are available for urine analysis, however they primarily detect leukocyte esterase as an indirect indicator of infection and cannot provide accurate bacteria count readings. Additionally, these dipsticks lack selectivity and may produce false-positive results due to the presence of other factors, including common medications such as antibiotics, aspirin, corticosteroids, and diuretics, which can also cause the appearance of leukocytes in urine.

Therefore, there continues to be a need for a rapid and user-friendly UTI sensor that can complement the streak plate method, particularly in settings such as outpatient clinics and point-of-care diagnostics. Preferably, the UTI sensor offers direct and selective bacteria quantification which is highly desirable in clinical settings.

### SUMMARY

In one aspect, a microfluidic device is described, said microfluidic device comprising:

an injection component comprising a first inlet and a first outlet communicatively connected to one another, wherein the first inlet is sized to accommodate a syringe and the first outlet is microfluidic in size;

a detection component comprising a second inlet and a second outlet communicatively connected to one another via a microfluidic channel, wherein the detection component further comprises an opening for an electrode sensor; and a lid, wherein the first outlet and the second inlet are substantially the same size and the injection component can be complimentarily connected to the detection component such that the first outlet and the second inlet are substantially aligned with one another, and wherein the detection component can be complimentarily connected to the lid.

In another aspect, a sensor system is described, said sensor system comprising a microfluidic device and an electrode sensor, wherein the electrode sensor comprises a substrate having a polymeric coating comprising an ionophore that binds a target ion to be sensed.

In still another aspect, a method of quantifying an amount of bacteria in a biofluid sample is described, the method comprising:
capturing bacteria from the biofluid sample on a filter membrane positioned in a microfluidic device;
rinsing the captured bacteria with water;
positioning an electrode sensor comprising a polymer coating thereon in the microfluidic device, wherein the polymer coating comprises a ionophore that binds a target ion to be sensed, wherein the target ion is depleted in the presence of bacteria;
introducing a solution comprising a known amount of the target ion to be sensed to the microfluidic device;
detecting a voltage loss of the target ion; and
quantifying the amount of bacteria in the biofluid sample.

Another aspect relates to a method of detecting a urinary tract infection (UTI) in a subject, said method comprising quantifying an amount of bacteria present in a biofluid of the subject using a sensor system, wherein if the amount of bacteria is greater than about 100,000 CFU/mL, the subject has a UTI.

Other aspects, features and embodiments of the invention will be more fully apparent from the ensuing disclosure and appended claims.

### BRIEF DESCRIPTION OF THE FIGURES

FIG. 1. A side view of an embodiment of the bacteria detection microfluidic device described herein comprises three 3D-printed components: the injection component 10, the detection component 20, and the lid 30.

FIG. 2. A top view of the three parts of FIG. 1.

FIG. 3. A cross-sectional view of the three parts of FIG. 1.

FIG. 4. A perspective view of an embodiment of an injection component 10.

FIG. 5. A transparent perspective view of the injection component 10 showing the fluid path therein.

FIG. 6. A cross-sectional view of the injection component 10 showing a fluid path therein.

FIG. 7. A perspective first-end view of the detection component 20.

FIG. 8. A perspective second-end view of the detection component 20.

FIG. 9. A first-end view of the detection component 20.

FIG. 10. A transparent perspective view of the detection component 20 showing a fluid path therein.

FIG. 11. A cross-sectional view (along a first plane) of the detection component 20 showing a fluid path therein.

FIG. 12. Another cross-sectional view (along a second plane) of the detection component 20 showing an opening for holding an electrode sensor therein.

FIG. 13. A perspective view of a lid 30.

FIG. 14. A top view of the connected components of FIG. 2.

FIG. 15. A cross-sectional view of the connected components of FIG. 3.

FIG. 16. Engineering sketch of an embodiment of the detection component. The provided figures represent an embodiment and are not intended to limit the detection component in any way.

FIG. 17. Engineering sketch of an embodiment of the injection component. The provided figures represent an embodiment and are not intended to limit the injection component in any way.

FIG. 18. An embodiment of the injection component 10 described herein, wherein a piece of membrane (0.2 μm pore size) was placed to cover the first outlet 14 of the injection component.

FIG. 19. The device comprising the injection component 10 and the detection component 20, with the filter membrane positioned between the first outlet of the injection component and the second inlet of the detection component, was connected to a syringe loaded with 1 mL of bacteria solution.

FIG. 20. Hoechst assay was used to compare bacteria counts without the filtration versus bacteria counts trapped by the membrane (N=3, error=stdev).

FIG. 21. An embodiment showing where the electrode sensor can be placed in the detection component of the microfluidic device.

FIG. 22. An image showing the placement of the electrode sensor in the detection component and the injection of the silver solution to the microfluidic device.

FIG. 23. A calibration curve of the electrode sensor over a range of silver concentrations (N=3, error=stdev).

FIG. 24. A example of a calibration curve between voltage difference and bacteria numbers using the sensor system described herein to detect bacteria. (N=3, error=stdev).

FIG. 25. Measurements of the same samples using the sensor system and the streak plate method were statistically the same (N=3, error=standard error of mean).

FIG. 26. A flowchart illustrating an embodiment of the method of using the microfluidic device described herein.

### DETAILED DESCRIPTION, AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s).” “include(s),” “having.” “has.” “can.” “contain(s),” and variants thereof, as used herein, are intended to be open-ended transitional phrases, terms, or words that do not preclude the possibility of additional acts or structures. The singular forms “a.” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

As used herein, “substantially” is intended to denote an allowance of no more than about 5%, or no more than 3%, or no more than 2%, or no more than 1%, relative to ideal.

As used herein, the term “microfluidic device” refers to a device comprising fluidic structures and internal channels having microfluidic dimensions.

As used herein, the term “means for detecting” or “detection means” refers to an apparatus for monitoring a signal and/or displaying signal value, e.g., to monitor the progress of an assay and/or to determine a result of an assay. A detection means may include a means for evaluation of a signal value. A signal may be detected and/or evaluated by a detection device able to measure potential (voltage), current, conductivity, impedance, and/or charge, and combinations thereof, as well known to the person skilled in the art.

As used herein, the term “biofluid” refers to a biological fluid (e.g., a body fluid, a bodily fluid). For example, in some embodiments, a biofluid is an excretion (e.g., urine, sweat, exudate) and in some embodiments a biofluid is a secretion (e.g., breast milk, bile). In some embodiments, a biofluid is obtained using a needle (e.g., blood, cerebrospinal fluid, lymph). In some embodiments, a biofluid is produced as a result of a pathological process (e.g., a blister, cyst fluid). In some embodiments, a biofluid is derived from another biofluid (e.g., plasma, serum). Exemplary biofluids include, but are not limited to, amniotic fluid, aqueous humor, vitreous humor, bile, blood, blood plasma, blood serum, breast milk, cerebrospinal fluid, chyle, chime, endolymph, perilymph, exudates, feces, female ejaculate, gastric acid, gastric juice, lymph, mucus (e.g., nasal drainage, phlegm), pericardial fluid, peritoneal fluid, pleural fluid, pus, rheum, saliva, sebum (e.g., skin oil), serous fluid, semen, smegma, sputum, synovial fluid, sweat, tears, urine, vaginal secretion, and vomit. In some embodiments, the biofluid comprises urine.

An improved sensing system is described herein, wherein the sensing system is designed to address the critical health issue of bacterial infections, particularly urinary tract infections.

The antibacterial properties of silver have long been recognized [Kędziora et al., 2018; Long et al., 2017]. Recent advancements in scientific research have shed light on the mechanisms behind silver's effectiveness against bacteria. It has been discovered that Ag⁺ can penetrate bacterial cells through specific outer membrane proteins, particularly the outer membrane protein F(OmpF). OmpF is a transmembrane protein with a trimeric B-barrel structure, weighing approximately 39 kD. Studies have demonstrated that Ag⁺ can rapidly enter bacterial cells within a timeframe of fewer than 30 minutes and interact with various intracellular components, including proteins and even nucleic acids [Jung et al., 2008]. Furthermore, some extracellular Ag⁺ can directly bind to the bacterial cell wall through charge interactions [Whitlow & Rice, 1985]. Based on these findings, a detection method and a sensor system is described herein wherein the final depleted concentration of Ag⁺ relative to the initial concentration of Ag⁺ added to a sample solution comprising bacteria is determined, providing valuable insights into the number of bacteria present in the sample.

Three-dimensional (3D)-printed microfluidics have gained significant popularity in research laboratories. This innovative technology allows for the creation of objects with customized shapes based on computer-aided design (CAD) or computed tomography (CT) scans. Compared to traditional fabrication methods, 3D printing offers several advantages, including rapid prototyping and customization capabilities. With 3D printing, modifications to the CAD file can be easily made to accommodate new designs and facilitate efficient printing.

Broadly, a microfluidic device for the detection of bacteria is described herein. The microfluidic device can be fabricated using 3D printing technology, offering the flexibility to customize its dimensions based on the CAD file. It should however be appreciated by the person skilled in the art that the microfluidic device can be fabricated using any means known in the art. To address the challenge of Ag⁺ precipitation caused by chloride ions (CI) present in biological fluids, e.g., urine, a compact microfluidic-based filtration setup is positioned within the microfluidic device to trap bacteria. The microfluidic device can be easily connected to a syringe containing the sample, enabling efficient bacteria trapping while simultaneously removing Cl.

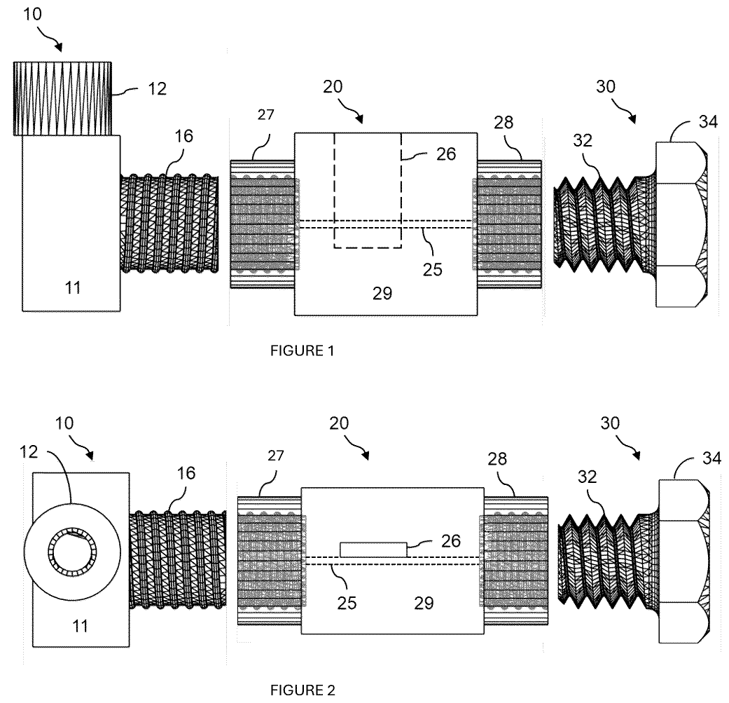

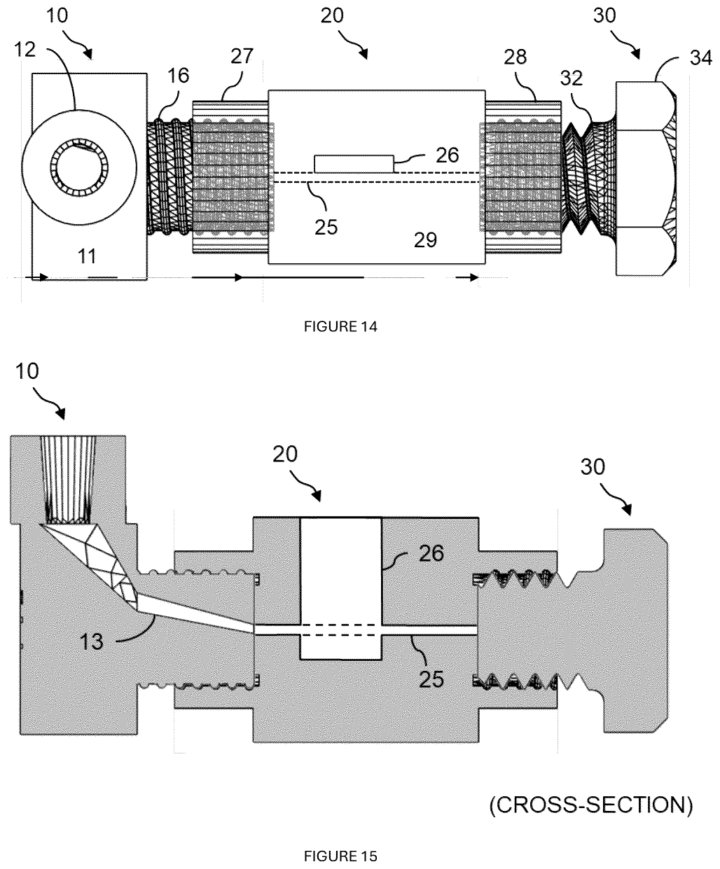

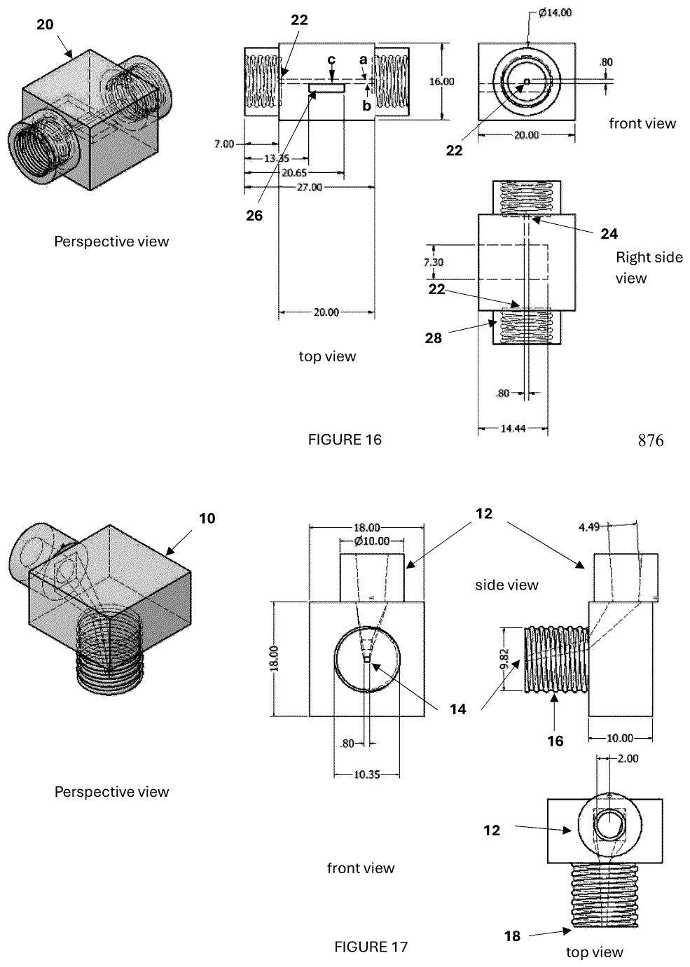

An embodiment of the microfluidic device is illustrated in FIGS. 1-3 (unconnected) and FIG. 14-15 (connected), with CAD drawings of two of the three components illustrated in FIGS. 16-17. As shown in the figures, the microfluidic device per se comprises three components: an injection component 100, a detection component 220, and a lid 30. As will be described further below, the sensor system comprises a microfluidic device, an ion-selective electrode sensor, and a detection device.

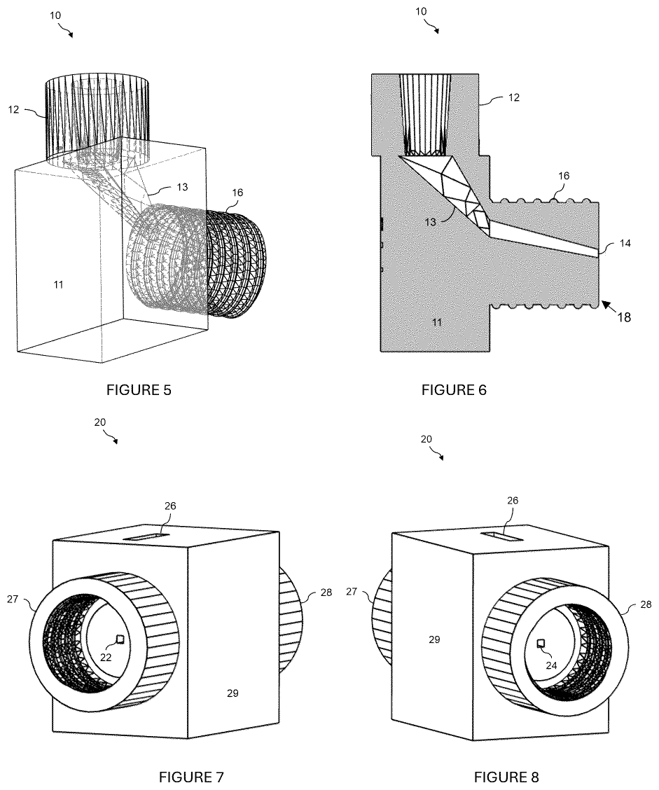

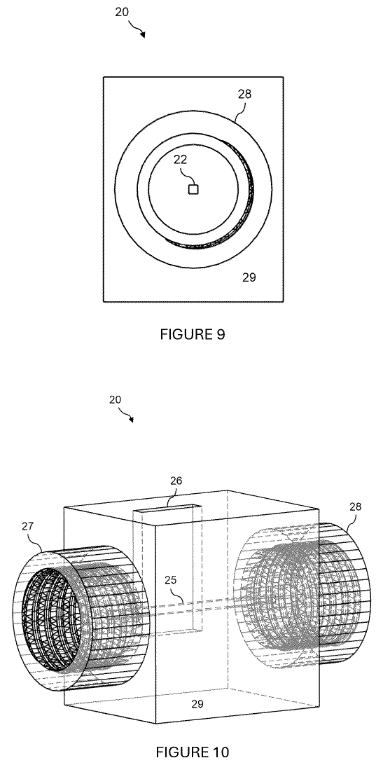

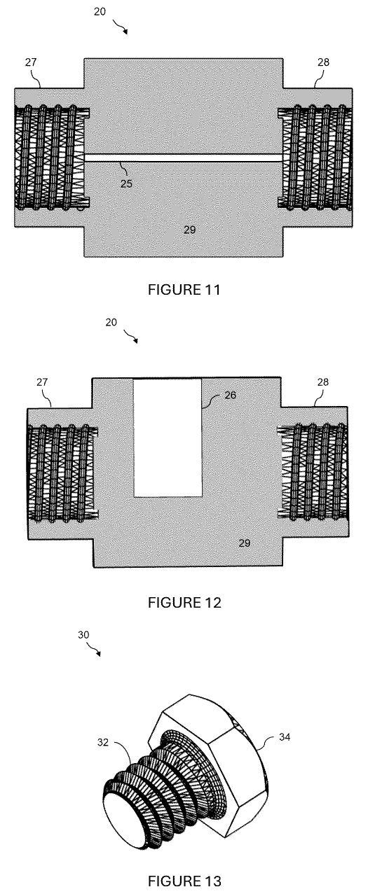

In a first aspect, a microfluidic device is described, said microfluidic device comprising an injection component 10, a detection component 20, and a lid 30, wherein the three components are sealingly connectable. In some embodiments, the injection component 10, for example as shown in FIGS. 4-6 and 17, comprises an inlet 12 that accommodates a syringe or equivalent thereof and an outlet 14 that has the width of a microfluidic channel, wherein the inlet and outlet are communicatively interconnected via channel 13. FIGS. 5-6 illustrate the transition of the width of the channel 13 from the inlet to the outlet, wherein the width of the channel at the inlet 12 accommodates a syringe but as the channel 13 transitions to the outlet 14, it becomes more and more narrow, eventually becoming microfluidic in width. It should be appreciated that the width of the channel 13 does not have to smoothly transition from wider to narrower (e.g., as illustrated in FIGS. 5-6) but instead the width of the inlet can transition to the width of the outlet over a shorter distance or even in a single step or several steps. In some embodiments, the outlet 14 is at an end of a first male fitting 16, wherein the first male fitting is threaded. In some embodiments, the end 18 of the first male fitting 16 is a substantially planar surface to accommodate a filter membrane (described below). In some embodiments, the detection component 20, for example as shown in FIGS. 7-12, and 16, comprises an inlet 22 and an outlet 24, wherein the inlet and outlet are communicatively interconnected by a microfluidic channel 25 having substantially the same size/width as the microfluidic channel at the outlet 14 of the injection component 10. In some embodiments, the inlet, the outlet, or both, is positioned in a female fitting, wherein a first female fitting 27 is complimentarily threaded so that the first male fitting 16 of the injection component 10 can be inserted and connected to the detection component 20 and the position of the microfluidic channel at the outlet 14 of the injection component 10 substantially aligns with the position of the microfluidic channel at the inlet 22 of the detection component 20. This permits fluid to enter the microfluidic channel 25 of the detection component 20 and fill at least a portion of the opening 26. Upon insertion of an electrode sensor in the an opening 26, the fluid in the opening 26 is in contact with a surface of the electrode sensor, and hence the fluid is in contact with a working electrode, a reference electrode, and a counter/amperometric electrode. In some embodiments, as illustrated in FIG. 16, the opening 26 is “offset” from the microfluidic channel 25, meaning that the opening 26 can have a first side “c” positioned at the edge of the microfluidic channel 25 (e.g., b=c, as shown), or the first side “c” can be positioned just within the microfluidic channel (somewhere between “a” and “b”). In some embodiments, but c #a, so that liquid from the microfluidic channel 25 can fill at least a portion of the opening 26 and contact a face of an inserted electrode sensor. The lid 30 is illustrated in FIG. 13 and comprises a second male fitting 32 that is complimentarily threaded so that the second male fitting 32 of the lid 30 can be inserted in a second female fitting 28, and connected to, the detection component 20.

It should be appreciated by the person skilled in the art that the means of connecting the three components is not limited to using threaded components and that other means are conceivable.

It should be appreciated by the person skilled in the art that although the injection component is shown and described as having a first male fitting, wherein the first male fitting is connected to a first female fitting of the detection component, it is within the skill of the art to have the injection component comprise a first female fitting that is connected to a first male fitting of the detection component instead. Similarly, the lid can comprise a second female fitting that is connected to a second male fitting of the detection component instead.

Microfluidic channels are known in the art. For the purposes of the instant application, the cross-sections of the microfluidic channels can be substantially square, substantially rectangular, substantially circular, triangular, polygonal, or substantially elliptical. In some embodiments, a microfluidic device comprises a microfluidic channel having microfluidic dimensions having an approximate cross-section in one dimension in a range of about 0.5 mm to about 1.5 mm, e.g., a square channel or a circular channel. For example, the microfluidic channel can have an approximate diameter or width of about 0.5 mm, 0.6 mm, 0.7 mm, 0.8 mm, 0.9 mm, 1 mm, 1.1 mm, 1.2 mm, 1.3 mm, 1.4 mm, 1.5 mm, or any range of these values. In some embodiments, the microfluidic channel can have an approximate diameter or width of about 0.7-0.9 mm. It should be appreciated that the approximate diameter or width of the microfluidic channel can be the consistent throughout the device, or can have varied dimensions, as understood by the person skilled in the art.

The microfluidic device can comprise, for example, and 3D printed polymer including, but are not limited to, acrylonitrile butadiene styrene (ABS), polycarbonate (PC), polylactic acid (PLA), high density polyethylene (HDPE), polyphenylsulfone (PPSU), poly(meth) acrylate, urethane acrylate, nylon, polyetherimide (PEI), polyether ether ketone (PEEK), high impact polystyrene (HIPS), thermoplastic polyurethane (TPU), polyamides (nylon), polytetrafluoroethylene (PTFE), derivatives of any of the species described herein, and combinations thereof. The chemical makeup of the microfluidic device is important. Specifically, each of the components of the microfluidic device (and the sensor system) must not react with the biofluid. In some embodiments, adhesives are not used. In some embodiments, the components of the microfluidic device are monolithic. In some embodiments, the components of the microfluidic device are not monolithic and must be glued together with adhesive prior to use. In some embodiments, the individual components of the microfluidic device can be sealed, for example, using complimentary threaded components. Other sealing means are readily understood by the person skilled in the art.

In some embodiments, the microfluidic device comprises an injection component, a detection component, and a lid, wherein the detection component and a portion of the injection component comprises a microfluidic channel.

In some embodiments, the microfluidic device comprises:
an injection component comprising a first inlet and a first outlet communicatively connected to one another, wherein the first inlet is sized to accommodate a syringe and the first outlet is microfluidic in size;
a detection component comprising a second inlet and a second outlet communicatively connected to one another via a microfluidic channel, wherein the detection component further comprises an opening for an electrode sensor; and
a lid,
wherein the first outlet and the second inlet are substantially the same size and the injection component can be complimentarily connected to the detection component such that the first outlet and the second inlet are substantially aligned with one another, and wherein the detection component can be complimentarily connected to the lid.

In some other embodiments, the microfluidic device comprises:
an injection component comprising a first inlet and a first outlet communicatively connected to one another, wherein the first inlet is sized to accommodate a syringe and the first outlet is microfluidic in size, wherein the injection component further comprises a first fitting;
a detection component comprising a second inlet and a second outlet communicatively connected to one another via a microfluidic channel, wherein the detection component further comprises a second fitting and a third fitting, wherein the detection component further comprises an opening for an electrode sensor; and
a lid comprising a fourth fitting,
wherein the first outlet and the second inlet are substantially the same size and the first fitting of the injection component can be connected to the second fitting of the detection component such that the first outlet and the second inlet are substantially aligned with one another, and wherein the fourth fitting of the lid can be connected to the third fitting of the detection component. In some embodiments, the first and second fitting are complimentarily threaded. In some embodiments, the third and fourth fitting are complimentarily threaded.

In some other embodiments, the microfluidic device comprises:
an injection component comprising a first inlet and a first outlet communicatively connected to one another, wherein the first inlet is sized to accommodate a syringe and the first outlet is microfluidic in size, wherein the first outlet is at an end of a threaded first male fitting;
a detection component comprising a second inlet and a second outlet communicatively connected to one another via a microfluidic channel, wherein the second inlet is positioned within a threaded first female fitting and the second outlet is positioned within a threaded second female fitting, wherein the detection component further comprises an opening for an electrode sensor; and
a lid comprising a second male fitting,
wherein the first outlet and the second inlet are substantially the same size and the first male fitting of the injection component can be connected to the first female fitting of the detection component such that the first outlet and the second inlet are substantially aligned with one another, and wherein the second male fitting of the lid can be connected to the second female fitting of the detection component.

In a second aspect, a sensor system is described, wherein the sensor system comprises the microfluidic device of the first aspect, and an electrode sensor, wherein the electrode sensor can be removably inserted into the opening of the detection component. In some embodiments, the sensor system further comprises a detection device.

In some embodiments, the electrode sensor comprises a substrate having a polymeric coating comprising an ionophore that binds the target ion to be sensed. In some embodiments, the electrode sensor has a three-electrode configuration, for example, as manufactured by BASi Research Products (West Lafayette, IN, U.S.A.), or the equivalent thereof, wherein the working, counter/amperometric and reference electrodes are screen-printed onto the substrate and all are located proximate to one another at a first end of the electrode sensor. The working, counter/amperometric and reference electrodes have integrated electrical contacts at a second end of the electrode sensor. The polymeric coating comprising the ionophore is obtained by introducing a solution comprising at least one plasticized polymer, at least one ion-exchanger. and at least one ionophore species dissolved in a solvent onto the working electrode of the electrode sensor. After the solvent is evaporated, the electrode sensor is ready for electrochemical measurements. It should be appreciated by the person skilled in the art that the electrode sensor is not limited to a three-electrode configuration. Any configuration of electrodes known in the art can be used with adaptations to the detection component.

In some embodiments, the solution comprises a cocktail in a solvent, wherein the cocktail comprises at least one polymer, at least one ionophore species, at least one plasticizer, and at least one cation-exchanger.

In some embodiments, the ionophore binds the target ion to be sensed. In some embodiments, the target ion is an antibacterial species, e.g., silver. In some embodiments, the ionophore is a Ag⁺ ion-specific ionophore such as 5-(4-dimethylamino-benzylidene) rhodanine, silver ionophore III, silver ionophore IV, silver ionophore VI, or silver ionophore VII. In some embodiments, the ionophore is 5-(4-dimethylamino-benzylidene) rhodamine. In some embodiments, the at least one ionophore species is present in the cocktail in an amount in a range from about 1 wt % to about 10 wt %, based on the total weight of the cocktail. In some embodiments, the at least one ionophore species is present in the cocktail in an amount in a range from about 2 wt % to about 9 wt %, based on the total weight of the cocktail. In some embodiments, the at least one ionophore species is present in the cocktail in an amount in a range from about 3 wt % to about 7 wt %, based on the total weight of the cocktail. In some embodiments, the at least one ionophore species is present in the cocktail in an amount in a range from about 4 wt % to about 6 wt %, based on the total weight of the cocktail. It should be appreciated that the ionophore species can be specific to a target ion other than silver, depending what the user wants to target.

In some embodiments, the at least one polymer comprises polyvinyl chloride (PVC), polyurethane, poly(tetrafluoroethylene), poly(methyl methacrylate), silicone rubber, perfluoropolymers, and combinations thereof. In some embodiments, the at least one polymer is PVC. In some embodiments, the at least one polymer is present in the cocktail in an amount in a range from about 25 wt % to about 45 wt %, based on the total weight of the cocktail. In some embodiments, the at least one polymer is present in the cocktail in an amount in a range from about 30 wt % to about 40 wt %, based on the total weight of the cocktail. In some embodiments, the at least one polymer is present in the cocktail in an amount in a range from about 32 wt % to about 37 wt %, based on the total weight of the cocktail.

In some embodiments, the plasticizer comprises dioctyl sebacate (DOS), 2-nitrophenyl octyl ether, 2-Nitrophenyl dodecyl ether, or [12-(4-Ethylphenyl) dodecyl] 2-nitrophenyl ether. In some embodiments, the plasticizer comprises DOS. In some embodiments, the at least one plasticizer is present in the cocktail in an amount in a range from about 45 wt % to about 75 wt %, based on the total weight of the cocktail. In some embodiments, the at least one plasticizer is present in the cocktail in an amount in a range from about 50 wt % to about 70 wt %, based on the total weight of the cocktail. In some embodiments, the at least one plasticizer is present in the cocktail in an amount in a range from about 55 wt % to about 65 wt %, based on the total weight of the cocktail.

In some embodiments, the cation-exchanger comprises sodium tetraphenylborate, tetrabutylammonium tetrabutylborate (TBA TBB), sodium tetrakis [3,5-bis(trifluoromethyl)phenyl] borate, potassium tetrakis [4-chlorophenyl] borate, potassium tetrakis [3,5-bis(trifluoromethyl)phenyl] borate (KTTFPB), or 1-methyl-3-n-octylimidazolium bis(trifluoromethylsulfonyl)imide (MeOctIm TFSI). In some embodiments, the at least one cation-exchanger is present in the cocktail in an amount in a range from about 0.1 wt % to about 2 wt %, based on the total weight of the cocktail. In some embodiments, the at least one cation-exchanger is present in the cocktail in an amount in a range from about 0.5 wt % to about 1.5 wt %, based on the total weight of the cocktail. In some embodiments, the at least one cation-exchanger is present in the cocktail in an amount in a range from about 0.8 wt % to about 1.2 wt %, based on the total weight of the cocktail.

In some embodiments, the solvent comprises at least one of tetrahydrofuran (THF) or cyclohexanone. The ratio of solvent to cocktail, by weight, is in a range from about 10:1 to about 15:1, or about 11:1 to about 14:1, or about 12:1 to about 14:1, or about 13:1 to about 14:1.

In some embodiments, the cocktail comprises about 25 wt % to about 45 wt % of at least one polymer, about 1 wt % to about 10 wt % of at least one ionophore species, about 45 wt % to about 75 wt % of at least one plasticizer, and about 0.1 wt % to about 2 wt % of at least one cation-exchanger, based on the total weight of the cocktail. In some other embodiments, the cocktail comprises about 25 wt % to about 45 wt % PVC, about 1 wt % to about 10 wt % 5-(4-dimethylamino-benzylidene) rhodanine, about 45 wt % to about 75 wt % DOS, and about 0.1 wt % to about 2 wt % sodium tetraphenylborate, based on the total weight of the cocktail.

In some embodiments, the solution comprises the cocktail and THE as the solvent, in a ratio of solvent to cocktail, by weight, in a range from about 10:1 to about 15:1.

In some embodiments, the solution is cast upon the working electrode of the electrode sensor and after the solvent is evaporated, the electrode sensor is ready for electrochemical measurements. In some embodiments, a method of making the electrode sensor is disclosed, said method comprising preparing the cocktail, combining the cocktail with a solvent to produce a solution, casting the solution onto a working electrode of an electrode sensor, and evaporating the solvent from the solution to produce an electrode sensor comprising an ionophore-containing polymer coating over the working electrode. The connection of the electrode sensor to a detection device such as a voltmeter is well known in the art.

As described herein, in some embodiments, the detection component of the microfluidic device comprises an opening 26 wherein the electrode sensor can be inserted in the opening 26 of the detection component 20 and the fluid in the microfluidic channel 25 of the detection component 20 will fill at least a portion of the opening 26 such that the fluid will be in contact with the ionophore-containing coating on the working electrode of the electrode sensor.

Accordingly, in a third aspect, a method of quantifying an amount of bacteria in a biofluid is described, the method broadly comprising capturing bacteria from the biofluid on a filter membrane positioned in a microfluidic device, e.g., of the first aspect, rinsing the captured bacteria, positioning an electrode sensor comprising an ionophore-containing polymer coating thereon in the microfluidic device, introducing a solution comprising a known amount of antibacterial species, e.g., Ag⁺, to the microfluidic device, detecting a voltage loss of the antibacterial species, and quantifying the amount of bacteria in the biofluid.

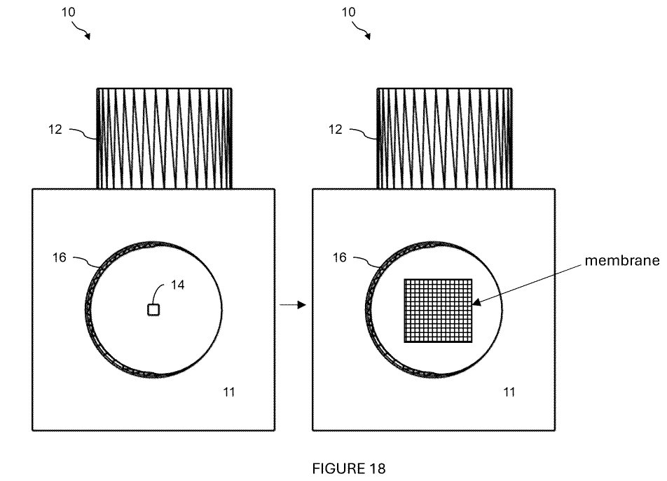

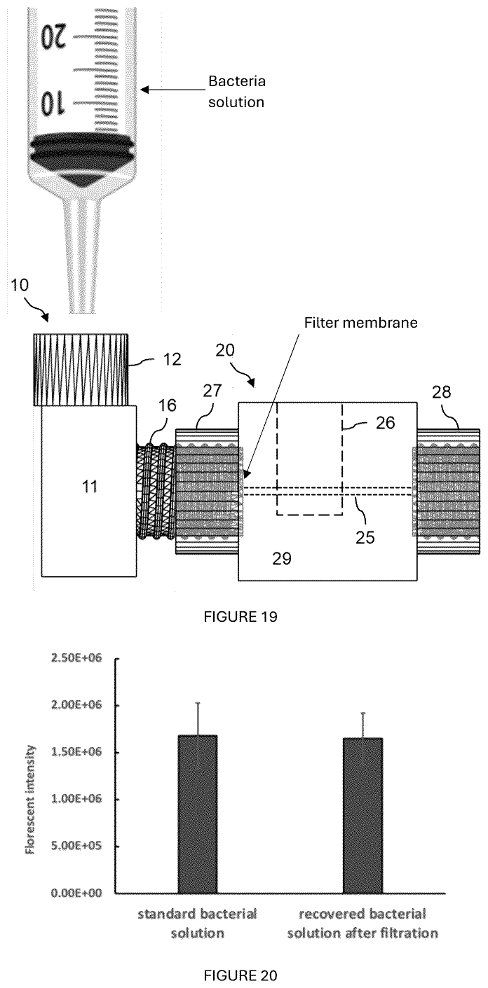

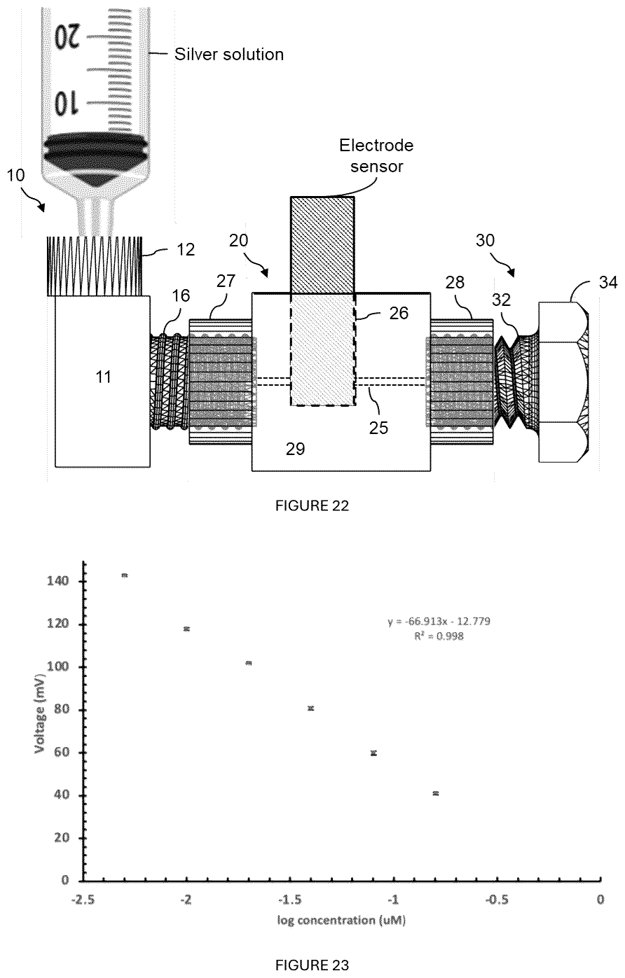

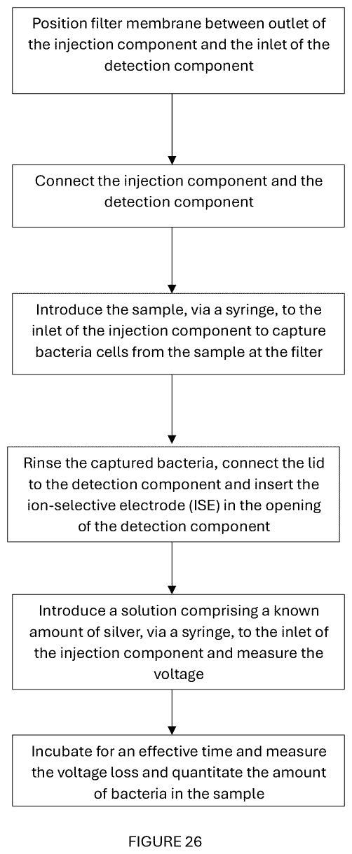

In order to perform the method of the third aspect, the bacteria of the biofluid must be captured by the microfluidic device. In one embodiment, as illustrated in the flowchart of FIG. 26, a filter membrane is positioned between the outlet 14 of the injection component 10 and the inlet 22 of the detection component 20 of the microfluidic device. As illustrated in FIG. 18, the filter paper is placed or affixed at the outlet 14 of the injection component at the substantially planar end 18 of the first male fitting 16. Thereafter the injection component 10 and the detection component 20 are connected, e.g., by screwing them together using threads, and the filter membrane is positioned between the outlet 14 of the injection component and the inlet 22 of the detection component of the microfluidic device (see, e.g., FIG. 19). Because of the substantial alignment of the microfluidic channel at the outlet 14 of the injection component 10 with the microfluidic channel at the inlet 22 of the detection component 20, the fluid that passes through the filter paper will enter the microfluidic channel 25 of the detection component (see, e.g., FIG. 15). Next, the biofluidic sample comprising bacteria is introduced to the inlet 12 of the injection component 10, the bacteria cells are captured by the filter membrane and liquid passes through to the microfluidic channel 25 of the detection component 20 and out the outlet 24. The liquid can be discarded. The bacteria cells captured by the filter membrane are washed by introducing water, via a syringe, to the inlet 12 of the injection component 10. The wash water passes through to the outlet 24 of the detection component 20 and can be discarded. In some embodiments, the opening of the detection component 20 is flushed with additional water. This ensures that any residual Cl ions are substantially removed to eliminate interference issues. The lid 30 is connected to the detection component 20, the ion-selective electrode sensor is inserted in the detection component 20, and a standard solution comprising an amount of an antibacterial species, for example, an amount of silver ions, is introduced to the inlet 12 of the injection component 10 (see, e.g., FIG. 22). A voltage reading is immediately taken to establish the initial voltage and hence concentration of antibacterial species, e.g., Ag⁺ ions, at time zero before any loss due to interaction with the bacteria occurs. The electrode sensor is then removed and the microfluidic device incubated for an effective time in a range of about 5 min to about 30 min at an effective temperature of about 30° C. to about 37° C. During this time, the antibacterial species of the standard solution interact with the captured bacteria and there is a depletion of the antibacterial species. At the end of the incubation period, the electrode sensor is reinserted into the opening 26 and the voltage loss, i.e., the final voltage minus the initial voltage, at the ion-specific electrode (ISE) sensor measured. Using a calibration curve, the final depleted concentration of antibacterial species, i.e., Ag⁺, relative to the initial concentration of antibacterial species, i.e., Ag⁺, introduced to the microfluidic device comprising captured bacteria is determined, providing an ability to quantify the number of bacteria present in the biofluid sample.

In some embodiments, the filter membrane efficiently separates bacteria cells from the biofluid, e.g., urine, thus eliminating the potential interference from Cl ions. In some embodiments, the pore size of the filter membrane is in a range from about 0.1 μm to about 0.2 μm, preferably about 0.2 μm. In some embodiments, the filter membrane comprises a polycarbonate membrane.

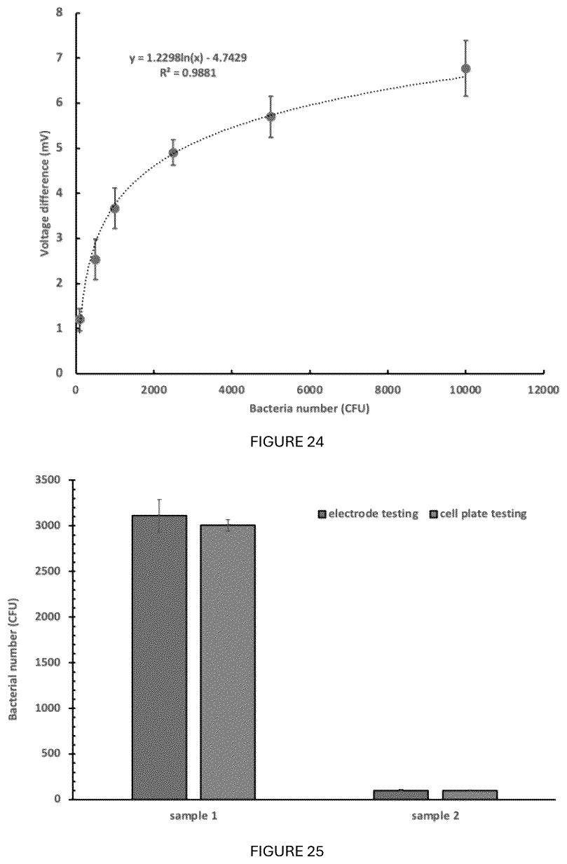

It should be appreciated that in order to determine the amount of bacteria present in the biofluid sample based on the voltage loss of the ion-selective electrode sensor requires the preparation of a calibration curve, as readily understood by the person skilled in the art. For example, one calibration curve can be prepared to determine the correlation between the electrode potential and the logarithm of the Ag⁺ concentration (e.g., as shown in FIG. 23). A second calibration curve can be prepared that compares the voltage difference and the bacteria number in CFU (e.g., as shown in FIG. 24).

In some embodiments, the antibacterial species is silver ions. By optimizing the Ag⁺ concentration, the sensor system described herein demonstrates remarkable sensitivity, detecting as few as 70 bacteria CFU/mL (limit of detection, LOD). In some embodiments, the optimized Ag⁺ concentration is in a range from about 1 μM to about 50 μM, or about 1 μM to about 30 μM, or about 1 μM to about 15 μM, or about 5 μM to about 10 μM. This sensitivity range aligns well with the diagnostic requirements for most infections.

In some embodiments, the limit of detection using the method described herein is as low as 70 bacteria CFU/mL.

Advantageously, the instant device and method eliminate the need for centrifugation to isolate bacterial cells from urine, as well as minimizing the need to perform a time-consuming streak plate analysis. Further, interfering ions such as Cl are washed away which enabled accurate and reliable results.

Accordingly, in some embodiments of the third aspect, the method of quantifying an amount of bacteria in a biofluid is described, the method comprising:
capturing bacteria from the biofluid on a filter membrane positioned in a microfluidic device;
rinsing the captured bacteria with water;
positioning an ion-specific electrode (ISE) sensor comprising an ionophore-containing polymer coating thereon in the microfluidic device, downstream of the filter membrane comprising the rinsed captured bacteria;
introducing a solution comprising an amount of antibacterial species to the microfluidic device and measuring the initial voltage at the ISE;
removing the ISE and incubating the microfluidic device for an effective amount of time at an effective temperature;
reinserting the ISE in the microfluidic device and measuring the final voltage at the ISE; and
using the change in voltage at the ISE, quantifying the amount of bacteria in the biofluid.

Accordingly, in some other embodiments of the third aspect, the method of quantifying an amount of bacteria in a biofluid is described, the method comprising:
capturing bacteria from the biofluid on a filter membrane positioned in a microfluidic device of the first aspect;
rinsing the captured bacteria with water;
positioning a silver ion-specific electrode (ISE) sensor comprising a silver ionophore-containing polymer coating thereon, as described in the second aspect, in the microfluidic device, downstream of the filter membrane comprising the rinsed captured bacteria;
introducing a solution comprising an amount of silver ions to the microfluidic device and measuring the initial voltage at the ISE;
removing the ISE and incubating the microfluidic device for an effective amount of time at an effective temperature;
reinserting the ISE in the microfluidic device and measuring the final voltage at the ISE; and
using the change in voltage measured at the ISE, quantifying the amount of bacteria in the biofluid.

In a fourth aspect, a method of detecting a urinary tract infection (UTI) in a subject is described, said method comprising quantifying an amount of bacteria present in a biofluid of the subject, for example as described in the third aspect, wherein if the amount of bacteria is greater than 100,000 CFU/mL, the subject has a UTI.

In some embodiments of the fourth aspect, a method of detecting UTIs in a subject is described, said method comprising capturing bacteria from a biofluid of the subject on a filter membrane positioned in a microfluidic device, e.g., of the first aspect, rinsing the captured bacteria, positioning an electrode sensor comprising an ionophore-containing polymer coating thereon in the microfluidic device, introducing a solution comprising a known amount of antibacterial species, e.g., Ag⁺, to the microfluidic device, detecting a voltage loss of the antibacterial species over time, and quantifying the amount of bacteria in the biofluid, wherein if the amount of bacteria is greater than 100,000 CFU/mL, the subject has a UTI.

The features and advantages of the invention are more fully illustrated by the following non-limiting examples, wherein all parts and percentages are by weight, unless otherwise expressly stated.

### EXAMPLE

### Experimental

### Chemicals and Materials

Magnesium chloride (MgCl₂), ammonium chloride (NH₄Cl), sodium sulfate (Na₂SO₄), polyvinyl chloride (PVC), bis(2-ethylhexyl) sebacate (DOS), sodium tetraphenylborate, urea, tetrahydrofuran (THF), and LB (Luria-Bertani) broth were purchased from Millipore-Sigma (MO, US). Sodium chloride (NaCl), calcium chloride (CaCl₂)), 5-(4-dimethylaminobezylidene) rhodanine, and silver nitrate (AgNO₃) were purchased from Alfa Aesar (MA, US). Polycarbonate membrane with 0.2 μm pore size was purchased from Whatman (PA, US). A three-electrode screen-printed electrode (SPE) was purchased from BASi (West Lafayette, IN, U.S.A.).

### 3D Printing of the Devices

The design of all components was carried out using Autodesk Inventor software (CA, US). Subsequently, the designed parts were fabricated using a 3D printer (Form 3B; Formlabs, MA, US). Formlabs Photopolymer Resin Clear (FLGPCL04) was selected as the printing material for these components. Following the printing process, the printed objects underwent a rinsing step with isopropanol for a duration of 30 minutes. To ensure proper curing, the printed objects were then exposed to UV light for a period of 1 hour.

### Ag⁺ electrode sensor coating

To create the Ag⁺ selective membrane on the surface of the electrode sensor, a polymer-containing solution was prepared. The polymer-containing solution comprised a cocktail comprising 5% 5-(4-dimethylamino-benzylidene) rhodanine as the ionophore, 60% dioctyl sebacate (DOS) as the plasticizer, 1% sodium tetraphenylborate as the cation-exchanger, and 34% polyvinyl chloride (PVC) as the polymeric matrix, with a total weight of 100 mg (all concentrations in weight/weight). The cocktail was mixed with 1.5 mL of tetrahydrofuran (THF) to produce the polymer-containing solution. A volume of 5 μL of the resulting polymer solution was carefully dropped onto a working electrode of the SPE electrode sensor. Subsequently, the electrode sensor was placed in a fume hood and left overnight to allow the THF solvent to completely evaporate, ensuring the drying of the polymer-containing solution thereon.

### Performance of Ag⁺ electrode sensor with Ag⁺ standards

A series of AgNO₃ standard solutions with concentrations of 6.25, 12.5, 25, 50, 100, and 200 μM were prepared, totaling 100 mL in volume. MilliQ water was used as the solvent for the preparation of these standards. The Ag⁺ ion selective electrode sensor was carefully inserted into the electrode sensor opening of the detection component of the microfluidic device. After an equilibration period of only 10 seconds, the potential reading on the electrode sensor stabilized, and this value was recorded. Following each reading, the electrode sensor was rinsed with MilliQ water three times to ensure the removal of any residual solution before the subsequent measurement. To generate calibration curves, the voltage readings were repeated three times for each standard solution. The average voltage values obtained for each concentration were plotted against the logarithm of the corresponding Ag⁺ concentrations. This plotting allowed for the creation of calibration curves, which served as a quantitative relationship between the detected voltages and the log concentrations of Ag⁺ in the standard solutions.

### Bacteria culture

Escherichia coli (E. coli) strain W-3104 was obtained from the American Type Culture Collection (ATCC. VA, U.S.A.). To initiate bacterial growth, the E. coli cells were inoculated into 15 mL of Luria-Bertani (LB) media and incubated in a humid incubator at 37° C. for a duration of 12 hours. Following the incubation period, 1 mL of the E. coli solution was subjected to centrifugation at 7000 rpm using an Eppendorf centrifuge (MA. US) for 3 minutes. The resulting cell pellet was then resuspended in 1 mL of water. For the quantification of bacterial numbers, 200 μL of the resuspended E. coli solution, along with an equal volume of a blank solution (composed of an identical buffer without any bacteria), were added to a 96-well plate. The absorbance of the solutions was measured at a wavelength of 600 nm (OD600) using a spectrophotometer.

### Performance of microfluidic filter system

A Hoechst assay was used to measure the total amount of DNA in the two lysates, so that the trapping efficiency could be determined. For the Hoechst assay, 200 μL of each bacterial lysate was dispensed into separate wells of a 96-well plate. Subsequently, 50 μL of a 10 μg/mL Hoechst assay solution (Hoechst 33258, ThermoFisher, PA, U.S.A.) was added to each well containing the lysate. A blank sample was also prepared by adding the lysis buffer without any bacteria. To detect the fluorescence signals, the plate was excited at a wavelength of 350 nm, and the emission was measured at 461 nm using a fluorescence reader. Prior to data analysis, the background fluorescence from the blank sample was subtracted from the fluorescence signals obtained from the Hoechst-DNA binding in the lysate samples. By comparing the resulting signals after subtracting the blank fluorescence, the fluorescence signals indicative of Hoechst-DNA binding were analyzed and compared among the bacterial lysate samples.

### Bacteria measurements

To prepare an artificial urine solution, the following concentrations of chemicals were mixed together: 34.1 mM NH₄Cl, 48 mM KCl, 4.4 mM CaCl₂), 3.2 mM MgCl₂, 102.8 mM NaCl, 16.2 mM Na₂SO₄, and 300 mM urea. This resulted in the formation of a standardized artificial urine solution. Bacterial solutions were prepared by diluting the stock culture in the artificial urine solution. A total of 1 mL of the diluted bacteria solution was loaded into a 3D-printed injection device. The bacteria solutions were then passed through the microfluidic device, which contained a filter membrane, to filter out the bacteria. Following filtration, 1 mL of water was pushed through the device to wash the filter membrane. After combining the lid with the microfluidic device, the Ag⁺ selective electrode sensor was inserted into the detection device. Subsequently, 200 μL of a 7.5 UM silver nitrate solution was added to the microfluidic device. The ion-selective electrode sensor provided an immediate reading of the Ag⁺ concentration. The electrode sensor was then removed, and the entire device was incubated at 37° C. for 15 minutes. After the incubation period, the Ag⁺ selective electrode sensor was placed back into the device to obtain a second reading. By comparing the amount of Ag⁺ taken up by the bacteria from the 7.5 μM silver nitrate solution, the number of bacteria in the sample could be determined. To ensure the accuracy of the data, the starting bacterial solution was also plated on streak plates. This was done by dissolving 1% (w/v) agar powder in LB broth. Subsequently, 10 mL of the agar solution was poured into a sterile Petri dish with a diameter of 60 mm. After the plates were allowed to solidify in a sterile hood at room temperature for 10 minutes, 100 μL of the bacterial solution was plated on the agar plate. The plates were then incubated overnight at 37° C. and the resulting colony numbers were counted as a reference for comparison with the results obtained from the Ag⁺ selective electrode sensor analysis.

### Results and Discussion

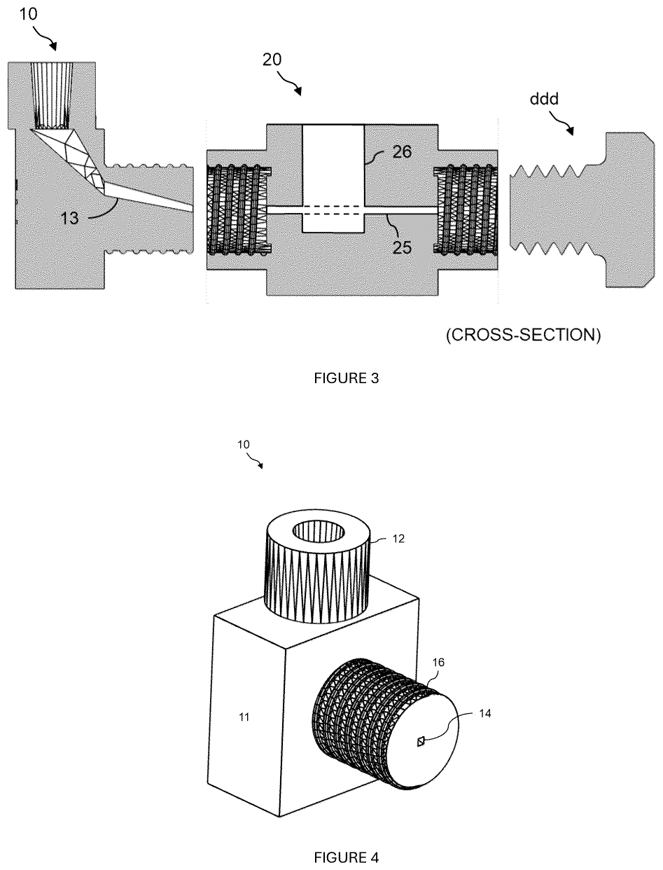

An embodiment of the bacteria detection microfluidic device described herein comprises three components: an injection device 10, a detection device 20, and a lid 30, as depicted in FIG. 1. The top view of the three components is shown in FIG. 2 and a cross-section of the three components is shown in FIG. 3. The microfluidic device was designed with three printed parts that could be combined, for example using printed threads. To visualize the fluid flow within the microfluidic device, a pigment solution was added at the injection device, and it was observed that the solution flowed through the entire system, filling at last a portion of the opening 26 the detection device 20. The lid successfully sealed the device, preventing any leakage. These three components could be easily assembled or disassembled depending on the specific experimental requirements. For the bacteria filtration process, it was necessary for the entire solution to pass through the microfluidic device as well as to wash it thoroughly. For example, upon combination of the injection device and the detection device, the first outlet 14 of the injection device 10 and the second inlet 22 of the detection device 20 align and the fluid can flow therethrough, as depicted in FIG. 15. On the other hand, for the silver detection process, a tight seal is required. After inserting the electrode sensor in the microfluidic device, all the components, including the injection component, the detection component, and the lid, needed to be assembled to ensure a leak-proof system, as shown in FIG. 15.

The modular design of the 3D-printed microfluidic device introduced herein offers significant advantages in terms of adaptability and versatility for various applications in bacteria detection and analysis. The CAD design details of the injection component and detection component are provided in FIGS. 16 and 17, respectively. It should be appreciated by the person skilled in the art that the components of the figures illustrated herein represent one embodiment of the microfluidic device described herein and are not intended to limit same. The skilled artisan can refer to the figures provided herein and reproduce or customize the system according to their specific needs. This feature allows laboratories with access to 3D printers to fabricate the devices in-house, ensuring full control over the manufacturing process and facilitating rapid prototyping. Moreover, for laboratories that do not have their own 3D printers, alternative options are available. Online 3D printing services, often referred to as “cloud-manufacturing,” can be utilized. These services allow customers to upload their CAD files, and the structures are then 3D printed by the service provider, who subsequently ships the printed parts back to the customer. This approach offers convenience and cost-effectiveness, particularly for research laboratories that do not have immediate access to 3D printing facilities.

In order to overcome the interference caused by chloride ions (CI) present in biofluids, e.g., urine, which have the ability to precipitate silver ions (Ag⁺), a filter membrane can be incorporated into the bacteria detection microfluidic device. The purpose of this filter membrane is to efficiently separate bacteria from the biofluid, e.g., urine, thus eliminating the potential interference from Cl ions. FIG. 19 illustrates the injection component 10 comprising a male fitting 16 that is sealingly joined to a female fitting 27 of the detection component 20, with the location of the filter membrane positioned between the first outlet 14 and the second inlet 22 when the two components are joined. As shown in FIG. 18, prior to assembly, the porous filter membrane with a pore size of 0.2 μm is placed over the first outlet 14 of the male fitting 16 of the injection component 10, effectively covering the entire microfluidic channel width. The male fitting 16 of the injection component 10 is then joined to the female fitting 27 of the detection component 20, e.g., by screwing the two components together, and the filter membrane will then be positioned at the juncture of the two components. It is widely acknowledged that solutions can be effectively sterilized by passing them through 0.2 μm filters, as bacteria cells are generally larger than the pore size and thus become trapped on the filter membrane surface. It should be appreciated that the filter membrane can be positioned inside a female fitting of a component instead and once a male fitting of a different component is joined to the female fitting, e.g., by screwing the two components together, the filter membrane will then be positioned at the at the juncture of the two components.

Once the filtration system was assembled, the bacterial solution was introduced, and the filtration process was initiated. As shown in the Figures, the injection component comprises an inlet that sealingly accommodates a syringe. As the solution passed through the microfluidic channel, the filter membrane acted as a filter, selectively trapping the bacteria while allowing the fluid to pass through (see, e.g., FIG. 19). The fluid that passed through can be discarded and the captured bacteria rinsed thoroughly with water. Following the filtration step, the trapped bacteria on the membrane were subjected to further analysis.

The implementation of the microfluidic device effectively addressed the issue of Cl interference and enabled accurate and reliable detection of bacteria within the biofluid. By incorporating this filtration step, the specificity and sensitivity of the bacteria detection microfluidic device was improved, allowing for precise quantification of bacterial load. This innovative approach provides a valuable tool for rapid and efficient bacteria detection, with potential applications in various diagnostic settings.

To evaluate the efficiency and accuracy of the bacteria trapping process facilitated by the microfluidic device, a series of experiments were conducted using a known bacterial concentration of 10000 CFU/mL. Following filtration, the filter membrane containing the trapped bacteria was carefully removed from the microfluidic device and submerged in a lysis buffer to extract the bacterial DNA. Additionally, an equivalent number of bacteria (10000 CFU) were separately lysed in the same lysis buffer composition as a control. To assess the success of the bacteria trapping process, the Hoechst assay was employed, wherein a fluorescent dye binds to DNA to measure the total amount of DNA present in the bacterial lysates. As depicted in FIG. 20, the fluorescence intensities of the recovered bacteria after filtration were found to be comparable to the standard control, indicating a nearly 100% efficiency in bacteria trapping using the filter membrane positioned in the microfluidic device described herein.

These results provide strong evidence for the effectiveness of the microfluidic device in selectively capturing and retaining bacteria while allowing the passage of other components present in the biofluid, e.g., urine. The nearly identical fluorescence intensities observed between the recovered bacteria and the standard control highlight the successful retention of bacterial DNA on the filter membrane, validating the reliability of the filtration process. The high trapping efficiency achieved by the microfluidic device demonstrates its potential as a robust tool for bacteria detection and analysis. By effectively removing unwanted substances, such as Cl ions, and selectively capturing bacteria, this system offers enhanced specificity and accuracy in bacterial quantification. Furthermore, the compatibility of the microfluidic filter with downstream DNA analysis techniques, such as the Hoechst assay, further enhances its utility in bacterial characterization.

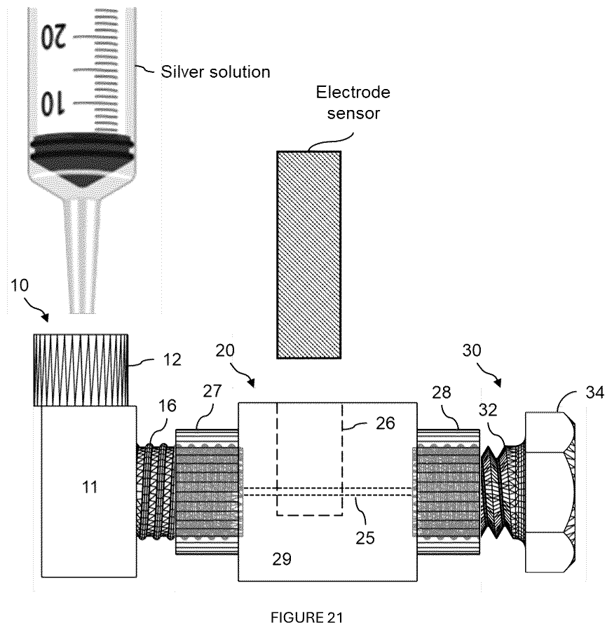

In FIGS. 21 and 22, a side view of the microfluidic device comprising the polymer-coated electrode sensor is shown, showcasing how the electrode sensor is positioned in the detection component of the microfluidic device. To evaluate the performance of the sensor system, a series of Ag⁺ standards were introduced (FIG. 22). The electrode sensor exhibited linear responses across a concentration range of 6.25 μM to 200 μM, as depicted in FIG. 23. The slope of the calibration curve generated from the electrode sensor was determined to be 66.9 mV, which closely approached the ideal slope of 59.2 mV predicted by the Nernst equation.

These results demonstrate the successful fabrication of an Ag⁺ ion selective electrode sensor using the 5-(4-dimethylaminobenzylidene) rhodanine ionophore embedded in a PVC membrane. The electrode sensor exhibited favorable sensitivity and a linear response over a wide range of Ag⁺ concentrations. The measured slope of the calibration curve indicates a good correlation between the electrode sensor potential and the logarithm of the Ag⁺ concentration.

These results highlight the suitability of the electrode sensor for accurate and reliable Ag⁺ detection in the bacteria detection microfluidic device described herein. Subsequently, a thorough assessment of the bacteria quantification capability of the microfluidic device was conducted. Bacteria standards were prepared in artificial urine and subjected to microfluidic filtration, followed by the addition of an Ag⁺ standard solution. After a 15-minute incubation period, the Ag⁺ loss was measured using the sensor system. Considering the limit of detection (LOD) of the Ag⁺ electrode sensor, which was determined to be 1.31 μM, and the limit of quantitation (LOQ) of 4.51 μM, an Ag⁺ standard concentration of 7.5 UM was selected for further analysis.

FIG. 24 presents the results obtained, illustrating a logarithmic relationship between the voltage loss (potential) and the bacteria numbers. Notably, a linear correlation was observed when plotting the voltage loss against the logarithm of the bacterial numbers. It is worth highlighting that the detection limit of our sensor system was approximately 70 CFU/mL of bacteria, which is sufficient for the detection of most infections. For instance, the diagnostic threshold for urinary tract infections is typically set at 1000 CFU/mL. To validate the accuracy of bacteria quantification using the sensor system, the results obtained from two unknown samples were compared using both the sensor and the streak plating method (FIG. 25). The measurements obtained from the two methods were found to be in close agreement, with no statistically significant differences observed. These findings further substantiate the accuracy of the sensor system in quantifying bacterial concentrations.

Overall, the characterization of the sensor system revealed its robust performance in bacteria quantification. The obtained logarithmic relationship between voltage loss and bacterial numbers, along with the linear correlation when plotting against the logarithm of bacterial numbers, demonstrate the reliable and precise quantification capability of the sensor system. Furthermore, the detection limit of 70 CFU/mL makes it suitable for practical applications, enabling the detection of a wide range of infections. The accuracy of the sensor system was further confirmed through the comparison with the well-established streak plating method, affirming its reliability as a tool for bacterial quantification.

### CONCLUSION

In this example, a cost-effective, portable, and user-friendly bacteria detection system that combines a 3D-printed microfluidic device and a custom-made silver ion-specific electrode sensor was introduced. The microfluidic device incorporates a filtration system with an 800 μm diameter channel, specifically designed to remove interfering ions present in biofluids, e.g., urine samples, and capture the bacteria. By optimizing the concentration of Ag⁺ (7.5 μM) used in the detection process, the sensor system demonstrates a remarkable sensitivity, capable of accurately detecting as few as 70 CFU/mL of bacteria. This detection limit surpasses the diagnostic threshold for urinary tract infections, which is typically set at 1000 CFU/mL. In summary, the innovative sensor system offers a highly sensitive and accurate approach for bacteria detection, with potential applications in various diagnostic settings involving bacteria infection.

### CLAUSES

Clause 1. A microfluidic device comprising:
   an injection component comprising a first inlet and a first outlet communicatively connected to one anoth r, wh r in the first inl t is siz d to accommodat a syring and the first out t is microfluidic in siz
   detection component comprising a second inlet and a second outlet communicatively connected to one another via a microfluidic channel, wherein the detection component further comprises an opening for an electrode sensor; and a lid,
   wherein the first outlet and the second inlet are substantially the same size and the injection component can be complimentarily connected to the detection component such that the first outlet and the second inlet are substantially aligned with one another, and wherein the detection component can be complimentarily connected to the lid.
Clause 2. The microfluidic device of any of the preceding clauses, wherein the injection component further comprises a first fitting, the detection component further comprises a second fitting and a third fitting, and the lid further comprising a fourth fitting, wherein the first fitting of the injection component can be connected to the second fitting of the detection component such that the first outlet and the second inlet are substantially aligned with one another, and wherein the fourth fitting of the lid can be connected to the third fitting of the detection component.
Clause 3. The microfluidic device of any of the preceding clauses, wherein the first fitting and the fourth fitting are threaded male fittings and the second fitting and the third fitting are threaded female fittings that are complimentary to the threaded male fittings of the first fitting and the fourth fitting.
Clause 4. The microfluidic device of any of the preceding clauses, further comprising a filter membrane positioned between the first outlet of the injection component and the second inlet of the detection component.
Clause 5. The microfluidic device of any of the preceding clauses, wherein the opening is offset from the microfluidic channel in the detection component, such that fluid entering the microfluidic channel can fill at least a portion of the opening.
Clause 6. A sensor system comprising the microfluidic device of any of the preceding clauses and an electrode sensor, wherein the electrode sensor comprises a substrate having a polymeric coating comprising an ionophore that binds a target ion to be sensed.
Clause 7. The sensor system of clause 6, wherein the target ion is silver (Ag⁺) and the ionophore is an Ag⁺ ion-specific ionophore.
Clause 8. The sensor system of clause 6, further comprising a detection device.
Clause 9. A method of quantifying an amount of bacteria in a biofluid sample, the method comprising:
   capturing bacteria from the biofluid sample on a filter membrane positioned in a microfluidic d [symbol] vic [symbol]
   rinsing th□captur [symbol] d bact [symbol] ria with wat [symbol] ;
   positioning an electrode sensor comprising a polymer coating thereon in the microfluidic device, wherein the polymer coating comprises a ionophore that binds a target ion to be sensed, wherein the target ion is d [symbol] pl [symbol] t [symbol] d in th□pr [symbol] [symbol] c □of bact [symbol] ria;
   introducing a solution comprising a known amount of the target ion to be sensed to the microfluidic d [symbol] vic [symbol]
   d□i□t cting a voltag □loss of th □targ [symbol] tion; and
   quantifying the amount of bacteria in the biofluid sample.
Clause 10. The method of clause 9, wherein the target ion is Ag⁺.
Clause 11. The method of clauses 9 or 10, wherein the electrode sensor is positioned downstream of the filter membrane comprising the rinsed captured bacteria.
Clause 12. The method of any of clauses 9-11, further comprising measuring an initial voltage at the electrode sensor immediately after introducing the solution comprising a known amount of the target ion to be sensed to the microfluidic device.
Clause 13. The method of clause 12, further comprising removing the electrode sensor after obtaining the initial voltage and incubating the microfluidic device for an effective amount of time at an [symbol] ff [symbol] ctiv □t [symbol] mp [symbol] ratur [symbol] and
   reinserting the electrode sensor in the microfluidic device and measuring the final voltage at the electrode sensor.
Clause 14. The method of any of clauses 9-13, wherein the amount of bacteria in the biofluid sample is determined relative to a calibration curve obtained using known concentrations of bacteria.
Clause 15. The method of any of clauses 9-14, wherein the polymer coating further comprises at least one polymer, at least one plasticizer, and at least one cation-exchanger.
Clause 16. The method of any of claims 9-15, wherein the microfluidic device is as recited in any of clauses 1-5.
Clause 17. A method of detecting a urinary tract infection (UTI) in a subject, said method comprising quantifying an amount of bacteria present in a biofluid of the subject using the method of any of clauses 9-16, wherein if the amount of bacteria is greater than about 100,000 CFU/mL, the subject has a UTI.
Clause 18. A method of detecting a urinary tract infection (UTI) in a subject, said method comprising quantifying an amount of bacteria present in a biofluid of the subject using the sensor system of any of clauses 6-8, wherein if the amount of bacteria is greater than about 100,000 CFU/mL, the subject has a UTI.

### REFERENCES

Jung, W. K.; Koo, H. C.; Kim, K. W.; Shin, S.; Kim, S. H.; Park, Y. H., Antibacterial activity and mechanism of action of the silver ion in Staphylococcus aureus and Escherichia coli. Appl Environ Microbiol 2008, 74 (7), 2171-8.
Kędziora, A.; Speruda, M.; Krzyżewska, E.; Rybka, J.; Łukowiak, A.; Bugla-Płoskońska, G., Similarities and Differences between Silver Ions and Silver in Nanoforms as Antibacterial Agents. Int J Mol Sci 2018, 19 (2), 444.
Long, Y.-M.; Hu, L.-G.; Yan, X.-T.; Zhao, X.-C.; Zhou, Q.-F.; Cai, Y.; Jiang, G.-B., Surface ligand controls silver ion release of nanosilver and its antibacterial activity against Escherichia coli. Int J Nanomedicine 2017, 12, 3193-3206.
Whitlow, S. I.; Rice, D. L., Silver complexation in river waters of central New York. Water Research 1985, 19 (5), 619-626.

What is claimed is:

1. A microfluidic device comprising:
an injection component comprising a first inlet and a first outlet communicatively connected to one another, wherein the first inlet is sized to accommodate a syringe and the first outlet is microfluidic in size;
a detection component comprising a second inlet and a second outlet communicatively connected to one another via a microfluidic channel, wherein the detection component further comprises an opening for an electrode sensor; and
a lid,
wherein the first outlet and the second inlet are substantially the same size and the injection component can be complimentarily connected to the detection component such that the first outlet and the second inlet are substantially aligned with one another, and wherein the detection component can be complimentarily connected to the lid.

2. The microfluidic device of claim 1, wherein the injection component further comprises a first fitting, the detection component further comprises a second fitting and a third fitting, and the lid further comprising a fourth fitting, wherein the first fitting of the injection component can be connected to the second fitting of the detection component such that the first outlet and the second inlet are substantially aligned with one another, and wherein the fourth fitting of the lid can be connected to the third fitting of the detection component.

3. The microfluidic device of claim 1, wherein the first fitting and the fourth fitting are threaded male fittings and the second fitting and the third fitting are threaded female fittings that are complimentary to the threaded male fittings of the first fitting and the fourth fitting.

4. The microfluidic device of claim 1, further comprising a filter membrane positioned between the first outlet of the injection component and the second inlet of the detection component.

5. The microfluidic device of claim 1, wherein the opening is offset from the microfluidic channel in the detection component, such that fluid entering the microfluidic channel can fill at least a portion of the opening.

6. A sensor system comprising the microfluidic device of claim 1 and an electrode sensor, wherein the electrode sensor comprises a substrate having a polymeric coating comprising an ionophore that binds a target ion to be sensed.

7. The sensor system of claim 6, wherein the target ion is silver (Ag⁺) and the ionophore is an Ag⁺ ion-specific ionophore.

8. The sensor system of claim 6, further comprising a detection device.

9. A method of quantifying an amount of bacteria in a biofluid sample, the method comprising:
capturing bacteria from the biofluid sample on a filter membrane positioned in a microfluidic device;
rinsing the captured bacteria with water;
positioning an electrode sensor comprising a polymer coating thereon in the microfluidic device, wherein the polymer coating comprises a ionophore that binds a target ion to be sensed, wherein the target ion is depleted in the presence of bacteria;
introducing a solution comprising a known amount of the target ion to be sensed to the microfluidic device;
detecting a voltage loss of the target ion; and
quantifying the amount of bacteria in the biofluid sample.

10. The method of claim 9, wherein the target ion is Ag⁺.

11. The method of claim 9, wherein the electrode sensor is positioned downstream of the filter membrane comprising the rinsed captured bacteria.

12. The method of claim 9, further comprising measuring an initial voltage at the electrode sensor immediately after introducing the solution comprising a known amount of the target ion to be sensed to the microfluidic device.

13. The method of claim 12, further comprising removing the electrode sensor after obtaining the initial voltage and incubating the microfluidic device for an effective amount of time at an effective temperature; and
reinserting the electrode sensor in the microfluidic device and measuring the final voltage at the electrode sensor.

14. The method of claim 9, wherein the amount of bacteria in the biofluid sample is determined relative to a calibration curve obtained using known concentrations of bacteria.

15. The method of claim 9, wherein the polymer coating further comprises at least one polymer, at least one plasticizer, and at least one cation-exchanger.

16. A method of detecting a urinary tract infection (UTI) in a subject, said method comprising quantifying an amount of bacteria present in a biofluid of the subject using the method of claim 9, wherein if the amount of bacteria is greater than about 100,000 CFU/mL, the subject has a UTI.

17. A method of detecting a urinary tract infection (UTI) in a subject, said method comprising quantifying an amount of bacteria present in a biofluid of the subject using the sensor system of claim 6, wherein if the amount of bacteria is greater than about 100,000 CFU/mL, the subject has a UTI.
